(19) United States
(12) Patent Application Publication
(10) Pub. No.: US 2026/0315058 A1
(43) Pub. Date: Oct. 15, 2026

(54) IMPROVEMENTS IN AND RELATING TO SURGICAL INSTRUMENTS, KITS AND METHODS OF USE

(71) Applicant: DEPUY IRELAND UNLIMITED COMPANY, Ringaskiddy Co. Cork (IE)

(72) Inventors: Peter MCMAHON, Hawthorn, Victoria (AU); Areeb HASSAN, Belrave South, Victoria (AU); Naomi TAYLOR, Coorparoo, Queensland (AU); Pierre GRAVEL, Hanover, MD (US); Edukondalu CHEMBETI, Hanover, MD (US); Vinay Kumar KOTAGI, Hanover, MD (US)

(21) Appl. No.: 19/484,613

(22) PCT Filed: May 16, 2024
(86) PCT No.: PCT/EP2024/063531

§ 371 Date: Nov. 14, 2025

(30) Foreign Application Priority Data
May 26, 2023 (GB) 2307934.6

(51) Int. Cl. A61B 17/02; A61B 17/00

(52) U.S. Cl. CPC A61B 17/0206; A61B 2017/00367

(57) ABSTRACT

A surgical instrument in particular a retractor configured to be manually operated by a user's thumb and fingers. In particular, a pair of handles may be grasped by the thumb and fingers and actuated relative to one another to exert force to first and second output elements to provide retraction of for example a surgical site.

### TECHNICAL FIELD

The present disclosure relates generally to surgical instruments, such as manually operated surgical instruments, kits including such instruments and methods of using such instruments. The surgical instruments have potential applicability in orthopaedic surgery, but are more generally applicable where the instrument applies force to sites on a patient, to another instrument or to another device, such as an implant.

### BACKGROUND

Joint arthroplasty is a well-known surgical procedure by which a diseased and/or damaged natural joint is replaced by a prosthetic joint. Examples include knee prosthesis and hip implants. During surgery, the procedure is made easier by using one or more retractors to increase access to the surgical site, and the bone therein, by spreading parts of the incision in the patent apart.

Such uses for retractors exist in a wide variety of surgical procedures.

In addition, the manual application and translation of forces through surgical instruments where parts of the instrument are forced together or forced apart is a feature of surgical instruments beyond retractors, such as scissors, forceps, clamps, staplers, laparoscopic instruments and the like.

### SUMMARY

According to a first aspect of the disclosure there is provided a surgical instrument, the surgical instrument including:
a first force input element and a second force input element, the first force input element and the second force input element being pivotally mounted relative to one another by a pivot, the first force input element being provided on a first side of the instrument and being provided in opposition to the second force input element provided on the second side of the instrument;
a first force output element and a second force output elements, the first force output element and the second force output element being pivotally mounted relative to one another by a pivot, the second force output element being provided on the first side of the instrument and being provided in opposition to the first force output element provided on the second side of the instrument.

The first force input element and the first force output element may be provided by a unitary element. The first force input element and the first force output element may have a fixed position for the first force input element relative to the first force output element. The first force input element may be provided free of any pivoting or rotating movement relative to the first force output element.

The second force input element and the second force output element may be provided by separate elements. The second force input element and the second force output element may have a non-fixed position for the second force input element relative to the second force output element. The second force input element may have at least a first position and a second position relative to the second force output element. The first position may be at a different angular position and/or rotational position and/or in a different configuration relative to the second position. The difference may be with respect to the second force output element.

The pivot for the first force input element and the second force input element may be a different pivot to the pivot for the first force output element and the second force output elements. The pivot for the first force input element and the second force input element may be a first pivot and the pivot for the first force output element and the second force output elements may be a second pivot.

The first pivot for the first force input element and the second force input element may be provided spaced apart, for instance along the length of the instrument.

The instrument may have a proximal end, for instance the force input element end. The instrument may have a distal end, for instance the force output element end.

The first pivot for the first force input element and the second force input element may be provided closer to the proximal end of the instrument than the second pivot for the first force output element and the second force output element. The second pivot for the first force output element and the second force output element may be provided closer to the distal end of the instrument than the first pivot for the first force input element and the second force input element.

The instrument may provide a mechanism between at least one of the force input elements and at least one of the force output elements. The instrument may provide a mechanism between only one of the force input elements and only one of the force output elements. The instrument may provide a mechanism between the second force input element and the second force output elements.

The mechanism may provide a link between the first pivot and the second pivot. The mechanism may convey force inputted about the first pivot to force outputted about the second pivot.

The mechanism may provide a force multiplier, for instance increased leverage, for the instrument. The mechanism may multiply the force inputted to a higher force outputted.

The mechanism may provide a distance multiplier, for instance increased travel distance. The mechanism may provide a distance travelled multiplier to a force output element compared with the distance travelled for the force input element.

The surgical instrument may be a retractor. The surgical instrument may be scissors or forceps or a clamp or a stapler or a laparoscopic instruments, where surgeon applied force is employed.

The surgical instrument may be manually operated, particularly with respect to the application of the input force.

The first force input element and the second force input element may have an expanded configuration and a contracted configuration. The separation of the first force input element and the second force input element being reduced in the contracted configuration compared with the expanded configuration. The separation of the first force input element and the second force input element being increased in the expanded configuration compared with the contracted configuration.

Transition of the first force input element and the second force input element to or towards the contracted configuration may increase the separation of a part, particularly the distal part, of the first force output element and second output element.

Transition of the first force input element and the second force input element to or towards the expanded configuration may not change the separation of a part, particularly the distal part, of the first force output element and second output element. The transition of the first force input element and the second force input element to or towards the expanded configuration may be promoted by one or more springs, such as a leaf spring.

Repeated cycles of transition of the first force input element and the second force input element to or towards the expanded configuration and then transition of the first force input element and the second force input element to or towards the contracted configuration may be used to increase and potentially maximise the separation of a part, particularly the distal part, of the first force output element and second output element.

The first force input element may be a part of a first arm of the surgical instrument, particularly a proximal section of the first arm. The first force input element may be a first handle element.

The first handle element may include a user grip, such as a ring grip, particularly at the proximal end. The user grip may be adapted to receive a thumb and/or one and/or two and/or three fingers.

The second force input element may be a second handle element.

The second handle element may include a user grip, such as a ring grip, particularly at the proximal end. The user grip may be adapted to receive a thumb and/or one and/or two and/or three fingers.

The first handle element may include a first abutment element. The second handle element may include a second abutment element. The first abutment element and the second abutment element may abut, particularly in the contracted configuration.

The first handle element may include a first arm section. The first arm section may extend between the user grip and a first pivot. The first handle element may include a mechanism mount, such as a plate extending sideways from the first handle element. The mechanism or a first part of the mechanism may be provided on the mechanism mount. The first handle element may be provided with a first mechanism housing part, for instance to enclose a first part of the mechanism.

The second handle element may include a second arm section. The second arm section may extend between the user grip and a first pivot. The second handle element may include a mechanism mount, such as a plate extending sideways from the second handle element. The mechanism or a second part of the mechanism may be provided on the mechanism mount. The second handle element may be provided with a second mechanism housing part, for instance to enclose a second part of the mechanism.

The first handle element may extend beyond the first pivot and/or beyond the first mechanism housing part. The first handle element may extend to a first force output element.

The second handle element may end at the first pivot and/or at the second mechanism housing part.

The first pivot may be a pivot for the first handle element and the second handle element relative to one another. One or both of the first force output element or the second force output element may not be pivot about the first pivot.

The first side of the instrument may the side with a first force input element and a first force output element. The first mechanism housing part may extend between the first side and the second side.

The second side of the instrument may be the side with the second force input element and the second force output element. The second mechanism housing part may extend between the second side and the first side.

The first pivot may be provided intermediate the first side and the second side.

The second pivot may be provided intermediate the first side and the second side. The first force output element and a second force output element may have a close state and an open state. The separation of the first force output element and the second force output element being reduced in the closed state compared with the open state. The separation of the first force output element and the second force output element may be increased in the open state compared with the closed state. The first output force element and the second force output element may have one or more intermediate states where the separation is greater than the closed state and/or less than the closed state.

Transition of the first force output element and the second force output element to or towards the open state may increase the separation of a part, particularly the distal part, of the first force output element and second output element.

Transition of the first force output element and the second force output element to or towards the closed state may decrease the separation of a part, particularly the distal part, of the first force output element and second output element.

The first force output element and the second force output element may transition from the closed state through one or more intermediate states to the open state.

The first force output element may be a part of a first arm of the surgical instrument, particularly a distal section of the first arm. The first force output element may be a first retractor arm.

The first retractor arm may include one or more retractor elements, particularly at the distal end. The retractor elements may extend away from the arm.

The second force output element may be a second retractor arm.

The second retractor arm may include one or more retractor elements, particularly at the distal end.

The retractor elements of the first retractor arm and the retractor elements of the second retractor arm may abut or be in proximity with one another, particularly in the closed state. The retractor elements of the first retractor arm and the retractor elements of the second retractor arm may be spaced from one another in the open state and/or one or more intermediate states.

The first arm, such as a first retractor arm, may include a first arm section and one or more retractor elements. The first arm section may extend between the second pivot and the distal end.

The second arm, such as a second retractor arm, may include a second arm section and one or more retractor elements. The second arm section may extend between the second pivot and the distal end. The second arm may include an interface with the mechanism, for instance a second mechanism part.

The second arm may include a projection, for instance beyond the second pivot location. The projection may extend proximally.

In an embodiment, the projection may extend proximally and sideways relative to the second pivot. The projection may provide the interface indirectly with the mechanism, such as a second part of the mechanism, for instance via a link element. The link element may be pivotally connected to the projection, for instance close to the proximal end thereof. The link may be pivotally connected to a pin. The pin may be provided by the mechanism, particularly a second part of the mechanism.

In an another embodiment, the projection may extend proximally on the other side of the second pivot compare with the second arm section bearing the distal end. The projection may provide the interface directly with the mechanism, such as a second part of the mechanism. The interface with the mechanism may comprise a slot which receives a pin. The slot may be provided by the second arm. The pin may be provided by the mechanism, particularly a second part of the mechanism.

The second pivot may be a pivot for the first output force element and the second output force element. The second pivot may be a pivot for a first arm, such as a first retractor arm, and a second arm, such as a second retractor arm. One or both of the first force input element or the second force input element may not be pivoted about the second pivot.

The first force input element and the first force output element may be formed of an integral element. The separation and/or position and/or angle of the proximal end of the first force input element and the distal end of the first output force element may be the same in the open state and/or the closed state and/or one or more intermediate states. The separation and/or position and/or angle of the distal end and the proximal end of the first force input element and the distal end of the first output force element may be the same in the expanded configuration and/or the contracted configuration and/or one or more intermediate configurations.

The second force input element and the second force output element may be formed of different and/or physically separate elements. The separation and/or position and/or angle of the proximal end of the second force input element and the distal end of the second output force element may be different in the open state and/or the closed state and/or one or more intermediate states. The separation and/or position and/or angle of the proximal end of the second force input element and the distal end of the second output force element may be different in the expanded configuration and/or the contracted configuration and/or one or more intermediate configurations.

The first pivot may be spaced from the second pivot by a distance of greater than 2 cm, for instance greater than 3 cm, potentially greater than 4 cm or even greater than 5 cm. The first pivot may be spaced from the second pivot by a distance of less than 10 cm, for instance less than 8 cm, potentially less than 6 cm or even less than 5 cm. The spacing may be measured along the long axis of the surgical instrument.

The first pivot and the second pivot may be provided on axes which are parallel to one another, but spaced from one another, particularly spaced along the long axis of the surgical instrument.

The mechanism may be provided as a first part of the mechanism and a second part of the mechanism.

The second part of the mechanism may be provided by the second force input element, particularly a second mechanism part housing, and a second drive element, such as a second pawl. The second drive element, such as a second pawl, may convey a drive force to the first part of the mechanism.

The first part of the mechanism may be provided by the first force input element, particularly a first mechanism part housing, and a first drive element, such as a cogwheel. The first drive element, such as a cogwheel, may convey a drive force from a second part of the mechanism to an interface, such as an interface with the second arm. The first drive element may convey the drive force through a pin extending from the first drive element. The pin may extend through the first mechanism part housing.

The mechanism may interface with the second arm, such as a second retractor arm, through a projection, for instance beyond the second pivot location. The projection may extend proximally.

In an embodiment, the projection may extend proximally and sideways relative to the second pivot. The projection may provide the interface indirectly with the mechanism, such as a first part of the mechanism, for instance via a link element. The link element may be pivotally connected to the projection, for instance close to the proximal end thereof. The link may be pivotally connected to a pin of the mechanism. The pin may be provided by the mechanism, particularly a first part of the mechanism.

In an another embodiment, the projection may extend proximally on the other side of the second pivot compare with the second arm section bearing the distal end. The projection may provide the interface directly with the mechanism, such as a first part of the mechanism. The interface with the mechanism may comprise a slot which receives a pin. The slot may be provided by the second arm. The pin may be provided by the mechanism, particularly a first part of the mechanism.

The mechanism may include a pivot, such as a pin, mounted on the second force input element, for instance a second mechanism part housing thereof. The mechanism may include a release mechanism, such as a release plate. The release plate may be mounted on the pivot. The release plate may be mounted between the second mechanism housing part and the first drive element. The mechanism may include a first drive element. The first drive element may be mounted on the pivot. The first drive element may be mounted between the release plate and the first mechanism part housing. The mechanism may include a pin on the first drive element. The mechanism may include a second drive element, such as a pawl, mounted on the second housing mechanism part. The mounting for the second drive element may allow rotation of the second drive element. The rotation may be promoted by a spring, such as a leaf spring.

The mechanism may include a second drive element, for instance a second pawl, that cooperates with a first drive element, such as a cogwheel to drive the first drive element. The cooperation may be provided in transition from the expanded configuration to or towards the contracted configuration. The cooperation may be removed in transition from the contracted configuration to or towards the expanded configuration.

The cooperation may be promoted by the rotation of the second drive element towards the first drive element. The cooperation may be removed by rotation of the second drive element away from the first drive element.

The mechanism may include a first locking element, for instance a first pawl. The first locking element may maintain the instrument in a given closed state and/or intermediate state and/or open state. The first locking element may engage with a first drive element to prevent movement, such as rotation, of the first drive element in a direction. The first locking element may allow movement, such as rotation, of the first drive element in a second different direction. The first locking element may be pivotally mounted, for instance on the first mechanism part housing. The first locking element may be promoted to rotate in a given direction, for instance into contact with the first drive element.

The pin may extend through a channel, for instance a channel in the first mechanism part housing. The channel may be curved. The channel may be an arc. The pin may be at one end of the channel in the closed state. The pin may be at the other end of the channel in the open state. The pin may be at an intermediate for an intermediate state.

In an embodiment, the channel may curve from a distal end to a proximal end.

In an another embodiment, the channel may curve from a first distal end, through a proximal mid-section to a second distal end.

A release mechanism may be provided. The release mechanism may include a rotatable arm, such as a lever arm. The release mechanism may extend from the mechanism housing. An end of the release mechanism may be accessible to the surgeon from the user grips. The release mechanism may include a release plate, for instance rotatable with the lever arm. The release plate may include one or more protrusions. A first protrusion may end in a first cam. The first cam may be adapted to move the first locking element out of cooperation with the first drive element, for instance by rotation of the cam. A second protrusion may end in a second cam. The second cam may be adapted to move the second drive element out of cooperation with the first drive element. The release mechanism may provide a released state. In a released state the instrument may be able to transition from an open state towards or to a closed state.

The first aspect may include any of the features, possibilities or options set out elsewhere in this document, including in the other aspects.

According to a second aspect of the disclosure there is provided a surgical kit, the surgical kit comprising a surgical instrument according to the first aspect of the disclosure and one or more additional surgical components.

The one or more surgical components may include one of more surgical instruments. The surgical instruments may be one or more of: different surgical retractors; different retractor blades; clamping instrument or devices; rakes; hooks; incision forming instruments.

The one or more surgical component may be an implant or a part of an implant, such as a femoral implant or hip implant.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature [s] being transferred to the intermediate component and the intermediate component feature[s] being transferred to the surgical instrument.

The second aspect may include any of the features, possibilities or options set out elsewhere in this document, including in the other aspects.

According to a third aspect of the disclosure there is provided a surgical method, the method comprising:
providing a surgical instrument according to the first aspect;
generating an incision in a patient;
increasing the access area through the incision by engaging the surgical instrument with the perimeter of the incision and transitioning the surgical instrument from a closed state to or towards an open state.

The surgical method may be an orthopaedic surgical method. The surgical instrument may be a retractor.

The method may include the first force input element and the second force input element may have an expanded configuration and a contracted configuration. The separation of the first force input element and the second force input element being reduced in the contracted configuration compared with the expanded configuration. The separation of the first force input element and the second force input element being increased in the expanded configuration compared with the contracted configuration.

The method may include transitioning from the expanded configuration to the contracted configuration.

Transition of the first force input element and the second force input element to or towards the contracted configuration may increase the separation of a part, particularly the distal part, of the first force output element and second output element.

The method may include transitioning from the contracted configuration to the expanded configuration.

Transition of the first force input element and the second force input element to or towards the expanded configuration may not change the separation of a part, particularly the distal part, of the first force output element and second output element.

Repeated cycles of transition of the first force input element and the second force input element to or towards the expanded configuration and then transition of the first force input element and the second force input element to or towards the contracted configuration may be used to increase and potentially maximise the separation of a part, particularly the distal part, of the first force output element and second output element.

The first force output element and a second force output element may have a close state and an open state. The separation of the first force output element and the second force output element being reduced in the closed state compared with the open state. The separation of the first force output element and the second force output element may be increased in the open state compared with the closed state. The first output force element and the second force output element may have one or more intermediate states where the separation is greater than the closed state and/or less than the closed state.

Transition of the first force output element and the second force output element to or towards the open state may increase the separation of a part, particularly the distal part, of the first force output element and second output element.

Transition of the first force output element and the second force output element to or towards the closed state may decrease the separation of a part, particularly the distal part, of the first force output element and second output element.

The first force output element and the second force output element may transition from the closed state through one or more intermediate states to the open state.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature [s] being transferred to the intermediate component and the intermediate component feature[s] being transferred to the surgical instrument.

The third aspect may include any of the features, possibilities or options set out elsewhere in this document, including in the other aspects.

### BRIEF DESCRIPTION OF THE DRAWINGS

Various embodiments of the disclosure will now be described, by way of example only, and with reference to the accompanying figures, in which:

FIG. 1 is an illustration of a prior art retractor;

FIG. 2 is an illustration of an embodiment of the retractor of the disclosure in a fully closed state and contracted handle configuration;

FIG. 3 is an illustration of the FIG. 2 embodiment, viewed from the other side, in the fully closed state and an expanded handle configuration;

FIG. 4 is an illustration of the FIG. 2 embodiment in a first intermediate state and contracted handle configuration;

FIG. 5a is an illustration of the FIG. 2 embodiment in a first intermediate state and expanded handle configuration;

FIG. 5b is an illustration of the FIG. 5a state and configuration, viewed for the other side;

FIG. 6 is an illustration of the FIG. 2 embodiment in a second intermediate state and contracted handle configuration;

FIG. 7a is an illustration of the FIG. 2 embodiment in a second intermediate state and expanded handle configuration;

FIG. 7b is an illustration of the FIG. 7a state and configuration, viewed for the other side;

FIG. 8 is an illustration of the FIG. 2 embodiment in a second intermediate state and contracted handle configuration;

FIG. 9a is an illustration of the FIG. 2 embodiment in a second intermediate state and expanded handle configuration;

FIG. 9b is an illustration of the FIG. 9a state and configuration, viewed for the other side;

FIG. 10 is an illustration of the FIG. 2 embodiment in a fully open state and expanded handle configuration;

FIG. 11a is an illustration of a second retractor embodiment in a fully closed state and expanded handle configuration;

FIG. 11b is an illustration of the FIG. 11a embodiment in a first intermediate state and expanded handle configuration;

FIG. 11c is an illustration of the FIG. 11a embodiment in a second intermediate state and expanded handle configuration;

FIG. 11d is an illustration of the FIG. 11a embodiment in a third intermediate state and expanded handle configuration;

FIG. 11e is an illustration of the FIG. 11a embodiment in a fourth intermediate state and expanded handle configuration;

FIG. 11f is an illustration of the FIG. 11a embodiment in a fully open state and expanded handle configuration;

FIG. 12a is a detailed view of the mechanism of the FIG. 11a to 11f embodiment from a first side in an expanded handle configuration;

FIG. 12b is a detailed view of the mechanism of the FIG. 11a to 11f embodiment from the same side in a contracted handle configuration; and

FIG. 13 is an illustration of hand size variation between people and genders.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

Surgical instruments, such as retractors, are used in a variety of surgical procedures, including in orthopaedic procedures, to apply force to sites on the patient or to another device or to an implant for a variety of purposes. In the case of a retractor, the force applied to the sites on the patient helps increase the separation of those sites and hence increase the surgical opening to assist with the surgical procedure.

In any surgical instrument where the force is manually generated by the hand of the surgeon, there is a direct link between the size of the surgeon's hand and the extent of the positional range the moveable parts of the surgical instrument can be moved between. There is also a direct link to the forces that can be applied to the moveable parts and hence translated through the instrument. In general, smaller hands have a reduced positional range and potentially a reduced level of force that can be applied. Smaller hands may also encounter increased fatigue levels seeking to operate such manual surgical instruments.

The embodiments of the disclosure seek to provide an approach to manual surgical instruments that are better attuned to users with smaller hands, including female surgeons. A reduction in the required input force for successful operation and/or reduced requirements for grip strength are also sought. Improvements in ergonomics for smaller hand users and the reduction of fatigue during use are also potential benefits of the approach.

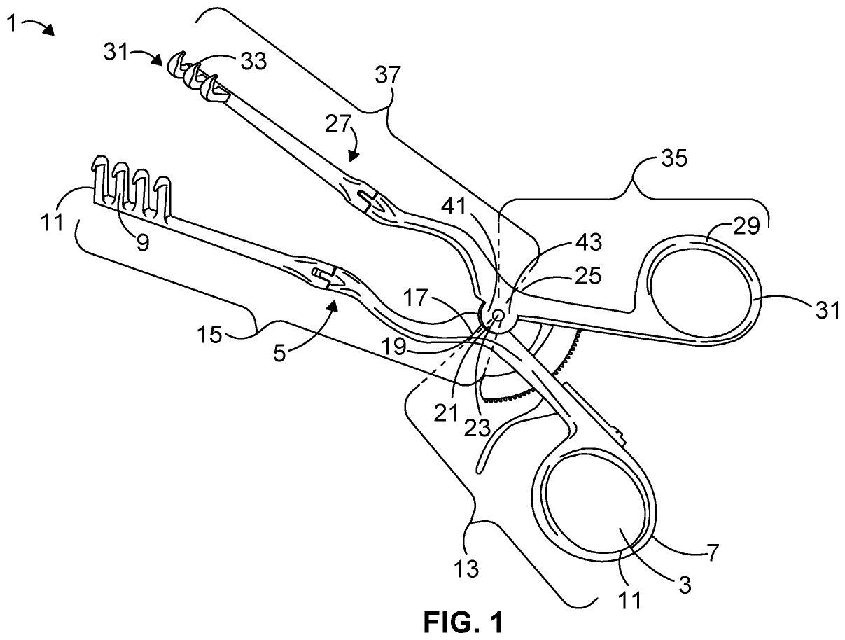

FIG. 1 is an illustration of a prior art retractor 1 provided with a first ring grip 3 on first arm 5. The first arm 5 is provided with the first ring grip 3 at a proximal end 7 and the retractor elements 9 at the distal end 11. A dog leg format is provided for the first arm 5 as a result of the proximal section 13 being angled relative to the distal section 15 at mounting location 17. The mounting location 17 features a protrusion 19 with a central aperture 21 which engages with a pin 23. The pin 23 provides a pivotal connection between the mounting location 17 of the first arm 5 and the mounting location 25 of the second arm 27.

The second arm 27 has a similar, but mirrored profile, compared with the first arm 5. Thus, the second arm 27 features a second ring grip 29 at a proximal end 31 and the second retractor elements 33 at the distal end 35. The first retractor elements 9 and the second retractor elements 33 oppose one another. A dog leg format is provided for the second arm 27 too, as a result of the proximal section 35 being angled relative to the distal section 37 at mounting location 39. The mounting location 25 features a protrusion 41 with a central aperture 43 which engages with the common pin 23.

The retractor 1 is shown in an intermediate state part way between a fully open state and a fully closed state. Movement of the first ring grip 3 and the second ring grip 29 towards one another will result in the spreading of the first retractor elements 9 and the second retractor elements 33 away from one another. Movement of the first ring grip 3 and the second ring grip 29 away from one another will result in the closing of the first retractor elements 9 and the second retractor elements 33 towards one another.

In the fully closed state, the separation between the first ring grip 3 and the second ring grip 29 is at a maximum. How this separation compares with the attainable separation of the surgeon's thumb from the surgeon's index [second] or the surgeon's first finger will determine whether the fully closed state can be reached and the force that the surgeon can apply in the immediate transition from the fully closed state to towards the intermediate state and then open state. If the surgeon is stretching or straining to reach that degree of separation then the force can be diminished and/or the comfort in operating can be impaired.

In the prior art retractor 1 of FIG. 1, the first arm 5 and the second arm 27 are unitary elements from their proximal to their distal ends and so the movement of the ring grips directly translates to the movement of the retractor elements. The leverage is defined by the relative position of the mounting locations, distally-proximally, and is set. A single translation of the ring grips provides all of the movement of the retractor elements.

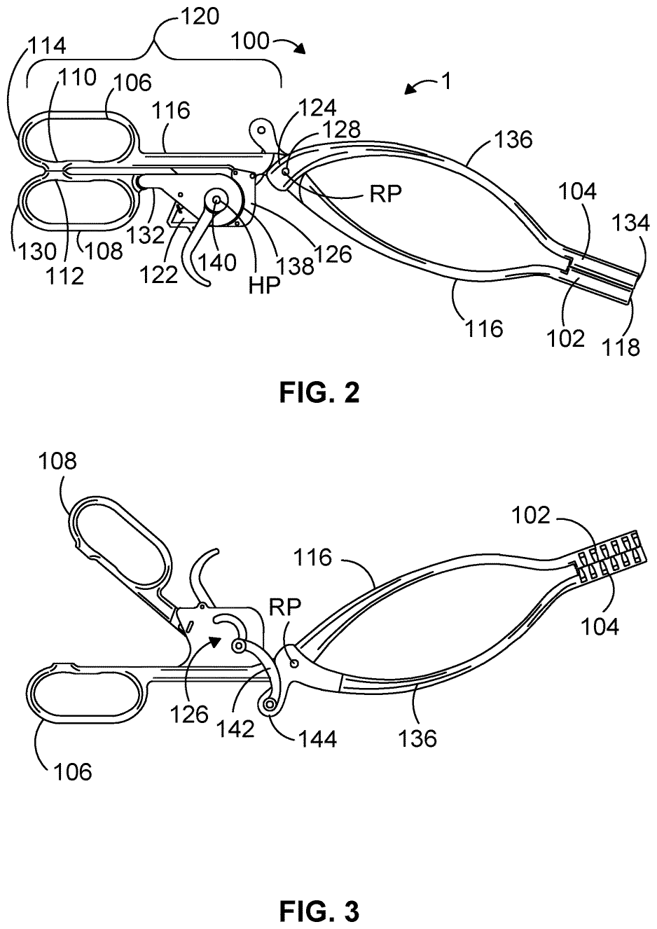

FIG. 2 is an illustration of an embodiment of the retractor 100 of the disclosure in a fully closed state and contracted handle configuration. The fully closed state is provided as the first retractor elements 102 and the second retractor elements 104 are abutting one another. The contracted handle configuration is provided as the first ring grip 106 and the second ring grip 108 are abutting one another. A first stop 110 on the first grip ring 106 and a second stop 112 on the second grip ring provide the abutment.

The fully closed state could be provided by the retractor elements and/or ring grips in proximity with one another, but constrained from moving closer to each other.

The first ring grip 106 and the second ring grip 108 are configured to receive either two fingers or the thumb of the surgeon and are configured to be useable by either hand in either orientation.

The first grip ring 106 is provided at the proximal end 114 of the first arm 116. The first arm 116 is a unitary element extending from the proximal end 114 to the distal end 118. The proximal section 120 of the first arm 116 is provided with a mechanism mount 122 extending away from the first arm 116. The first arm 116 also provides a first arm mount 124. The first arm mount 124 is distal of the mechanism mount 122. The first arm mount 124 provides the location at which the first arm 116 is connected to the second retractor arm 136.

The mechanism mount 122 has a first part of a mechanism 126 provided on it. An example of the mechanism 126 is describe in greater detail for a second embodiment of the disclosure. The mechanism mount 122 is in the form of a plate extending laterally from the first arm 116.

The first arm mount 124 is formed of a through aperture extending through the first arm 116, with the through aperture adapted to receive a pin 128 and so provide the retractor pivot location RP.

The second grip ring 108 is provided at the proximal end 130 of a second handle arm 132. The second retractor elements 104 are provided at the distal end 134 of a second retractor arm 136. Unlike the prior art and the first arm 116, the second handle arm 132 and the second retractor arm 136 are separate elements from each other.

The second handle arm 132 extends from the proximal end 130 to an intermediate end 138. The second handle arm 132 is pivotally mounted on the mechanism mount 122 at location HP. The intermediate end 138 of the second handle arm is provided with a second part of the mechanism 126. A pin 140 pivotally mounts the second handle arm 132 on the mechanism mount 122 at location HP and hence allows the proximal section 120 of the first arm 116 and the second handle arm 132 to pivot relative to one another. This pivot HP is separate from and spaced from the pivot RP for the distal section of the first arm 116 and the second retractor arm 136. The pivot RP is distal of the mechanism mount 122 and of the pivot HP.

The second retractor arm 136 is provided with a through aperture extending through the second retractor arm 136 which receives the pin 128 and hence provides the pivot RP.

FIG. 3 is an illustration of the FIG. 2 embodiment, viewed from the other side, in the fully closed state still, but transitioned to an expanded handle configuration. The transition to the expanded handle configuration is achieved by the surgeon increasing the separation of their thumb, in one of the first ring grip 106 and second ring grip 108, relative to their fingers, in the other of the second ring grip 108 and first ring grip 106. The mechanism 126 is in a free rotation configuration during rotation in this direction. As a result, the mechanism 126 does not translate any movement to the first arm 116 and/or second retractor arm 136 during the contracted handle configuration to expanded handle configuration. Once in the expanded handle configuration, the instrument is ready to start spreading the first retractor elements 102 and the second retractor elements 104 away from each other.

FIG. 3 also illustrates the link element 142 that connects the mechanism 126 to the second retractor arm 136 and in particular to a protrusion 144 thereon. The protrusion 144 extends away from the pivot RP and applies leverage to the second retractor arm 136 relative to the first arm 116 during spreading.

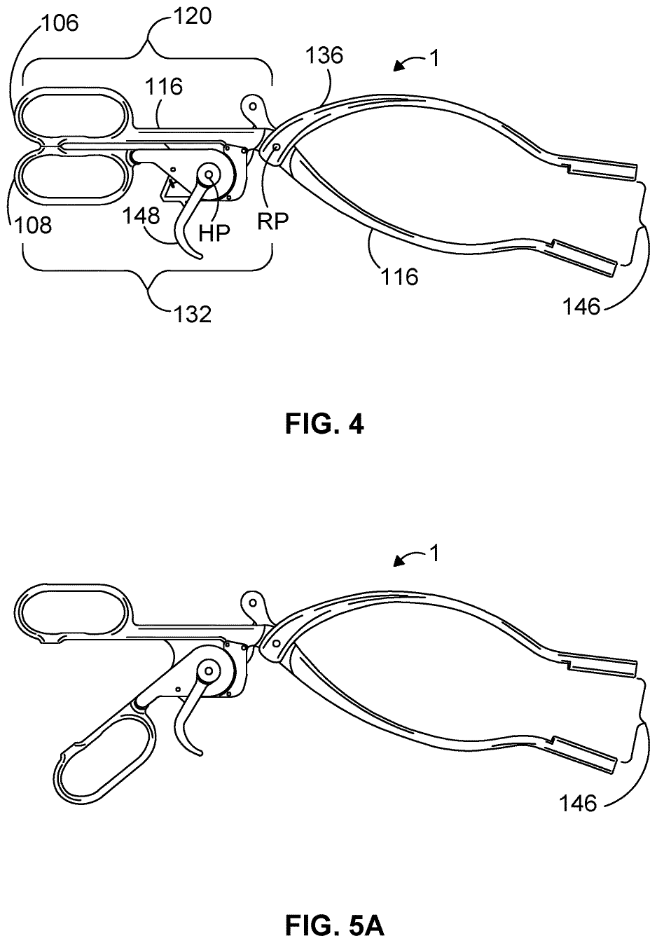

From the closed state and expanded handle configuration shown in FIG. 3, the surgeon squeezing the first ring grip 106 and second ring grip 108 together causes the retractor 1 to transition to the positions shown in FIG. 4. Squeezing the first ring grip 106 and second ring grip 108 together causes relative rotation of the proximal section 120 of the first arm 116 to the second handle arm 132. In FIG. 4 the retractor 1 is now in a first intermediate state, in particular with respect to the spreading, and once again in the contracted handle configuration. As can be seen, first intermediate spreading gap 146 has been provided.

Because the second handle arm 132 is non-unitary with the second retractor arm 136, the necessary maximum spread to minimum spread of the first ring grip 106 and the second ring grip 108 relative to one another can be brought within a range of movement that is comfortable and readily achieved by all users. There is no need to push the maximum spread value to a high level, to get a given degree of spreading at the distal ends, but with the result that the retractor cannot be comfortably and/or fully used by those surgeons with smaller hands.

Furthermore, because the movement to actuate the spreading can be feed through the mechanism 126, that mechanism 126 can be used to provide mechanical advantage to the spreading. Thus, increased spreading force can be obtained whilst maintaining the force that needs to be inputted by the surgeon within readily attainable and comfortable levels for all users.

The splitting of the pivot HP for the proximal section 120 of the first arm 116 relative to the second handle arm 132 away from the pivot RP for the distal section of the first arm 116 and the second retractor arm 136 assists with the deliver of both these benefits.

The mechanism 126 includes a holding mechanism, described in more detail below, which maintains the retractor 1 with the achieved spreading fap 146, even when the surgeon no longer applies the squeeze force to the first ring grip 106 and second ring grip 108. This prevents fatigue of the hand and arm in continuing to apply the force to maintain the spreading gap 146. It also allows a leaf spring or the like mounted on the retractor to spread the ring grips apart to the expanded handle state, once the squeeze force is removed, allowing for the next cycle of increasing the spreading gap to be achieved. The cycling of the grip rings from contracted to expanded to contracted to expanded and so on allows each cycle to increase the spreading gap. Thus, one single cycle does not have to achieve the full range of desired spreading gap for the retractor 1. Multiple low force, low distance travel for the thumb and fingers, cycles can be used to build up the spreading gap to the level sought. Within any one cycle, the spreading gap can be controlled at a partially increased level by only partially squeezing the grip rings together.

When release of the retractor 1 is desired to reduce or remove the spreading gap, then the release lever 148 can be pulled proximally by the surgeon and this releases the restraint within the mechanism 126. Further details of the release lever 148 are provided below.

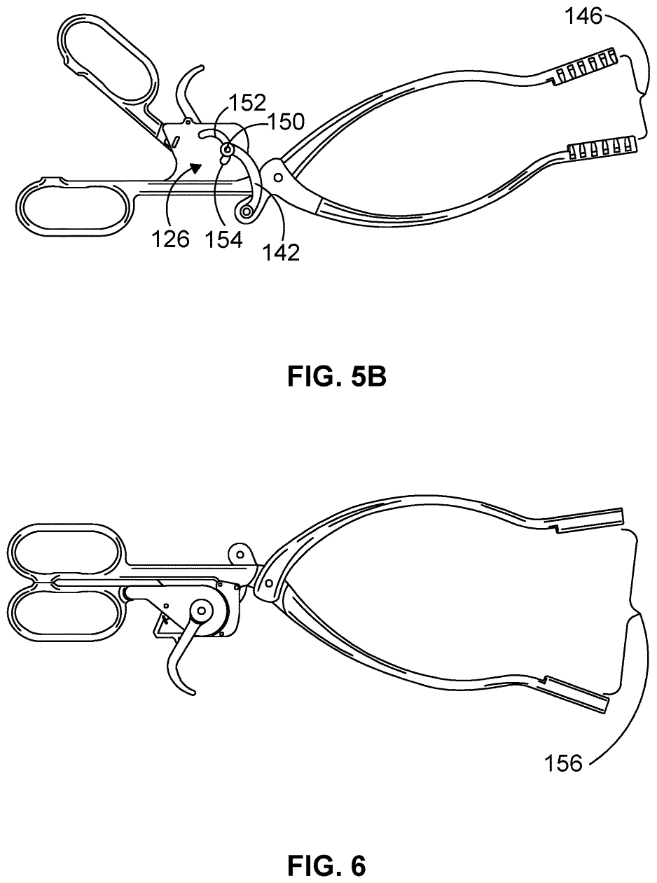

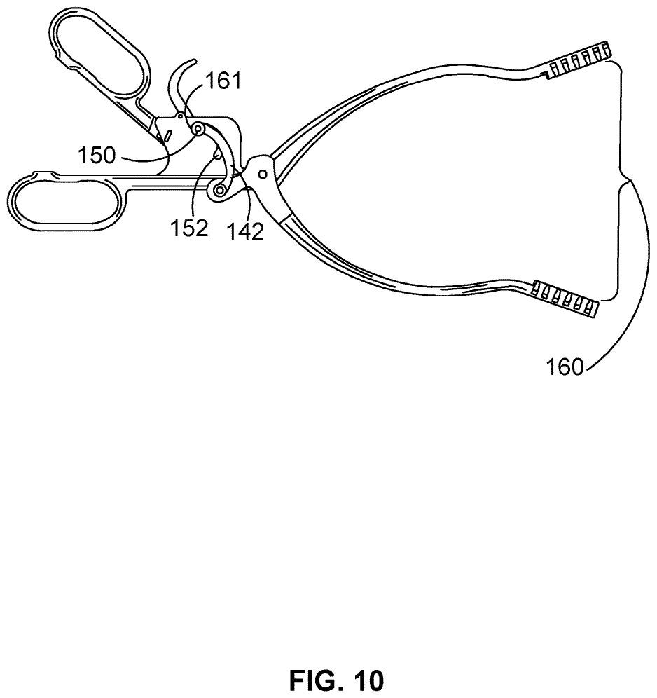

In general, FIGS. 5a, 5b, 6, 7a, 7b, 8, 9a, 9b and 10 show the further transitions to the fully open and spread form in FIG. 10. As such, these figures are briefly described with respect to the changes occurring for the same components as described in more detail above.

In FIG. 5a the retractor 1 is in a first intermediate state, similar to FIG. 4, but now with the expanded handle configuration ready for the next transition to increase spreading. As before, the mechanism 126 allows rotation from the contracted handle configuration to the expanded handle configuration without impacting upon the spreading gap 146 value.

FIG. 5b is an illustration of the FIG. 5a state and configuration, viewed for the other side and showing the proximal end 150 of the link element 142 partially advanced within curved channel 152 away from the closed state end 154.

FIG. 6 shows the increased spreading gap 156 resulting from the return of the ring grips to the contracted handle configuration; a second intermediate state for the retractor 1.

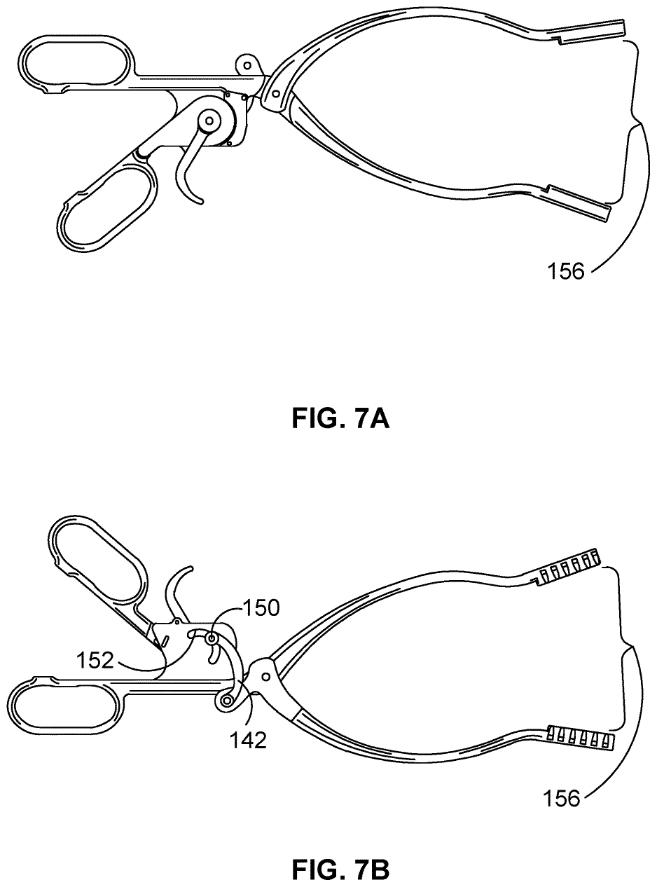

FIG. 7a is an illustration of the same increased spreading gap 156 present, but with the expanded handle configuration ready to activate the next cycle of spreading. FIG. 7b is an illustration of the FIG. 7a state and configuration, viewed for the other side. The change in position for the proximal end 150 of the link 142 within the channel 152 can be seen when compared with FIG. 5b.

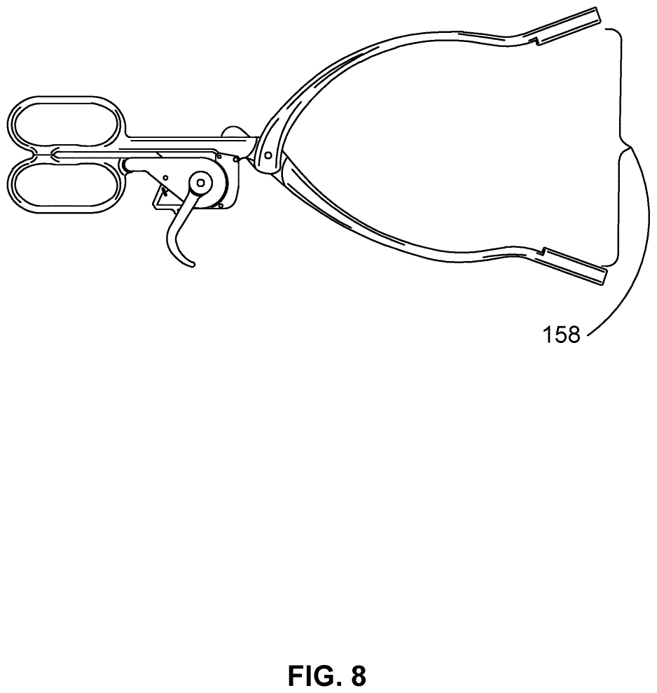

FIG. 8 shows the increased still further spreading gap 158 resulting from the return of the ring grips to the contracted handle configuration; a third intermediate state for the retractor 1.

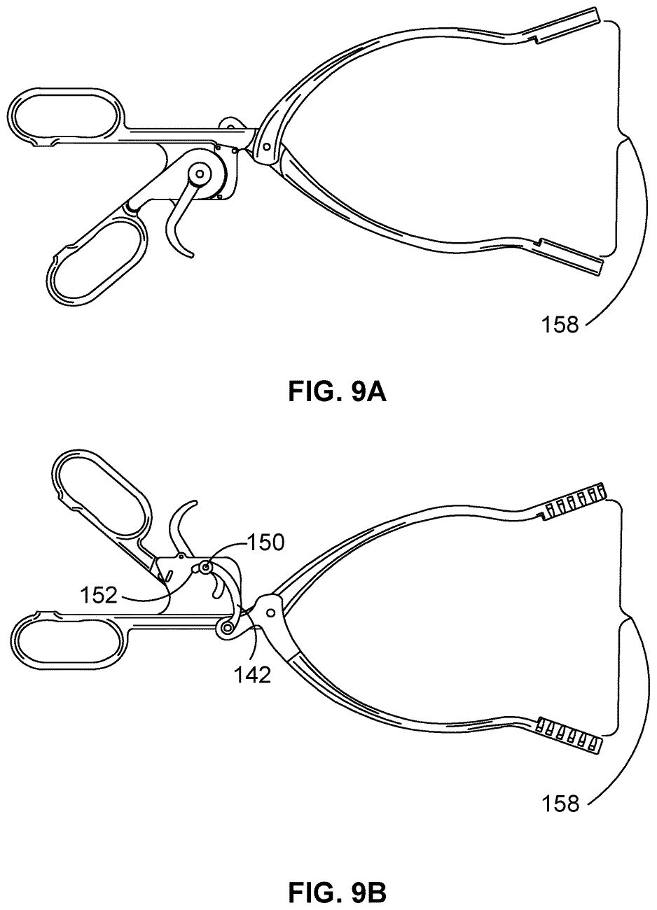

FIG. 9a is an illustration of the same increased spreading gap 158 present, but with the expanded handle configuration ready to activate the next cycle of spreading. FIG. 9b is an illustration of the FIG. 9a state and configuration, viewed for the other side. The change in position for the proximal end 150 of the link 142 within the channel 152 can be seen when compared with FIG. 7b.

FIG. 10 is an illustration of the end point with the fully open state achieved. The spreading gap 160 is larger again than the previous spreading gap 158. The proximal end 150 of the link 142 has reached the end 161 of the channel 152 and so further spreading is not possible. The retractor can be placed in the expanded handle configuration, as shown, but further squeezing together of the ring grips does not give increased spreading.

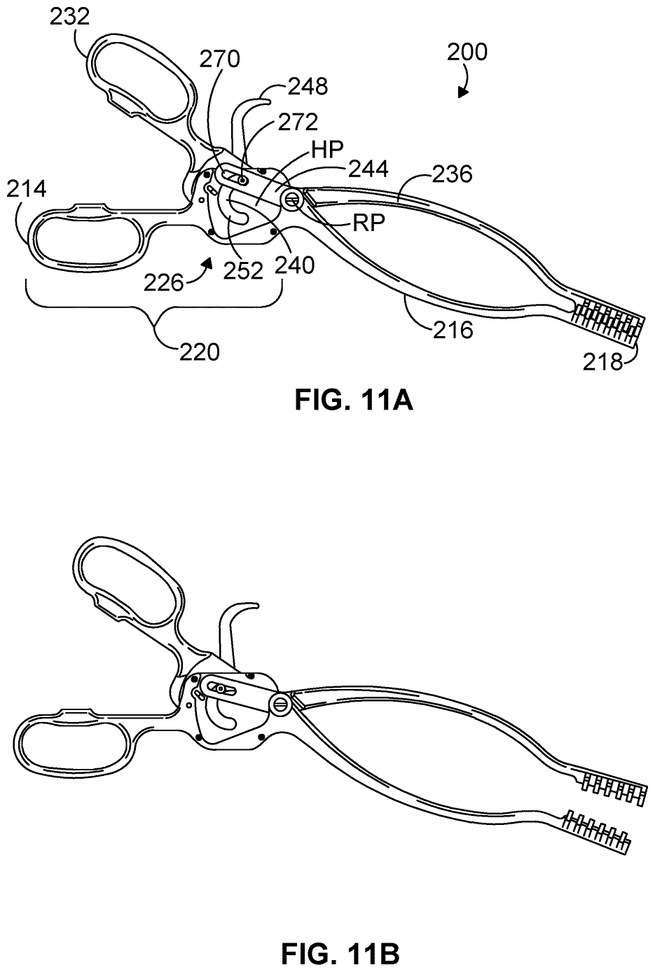

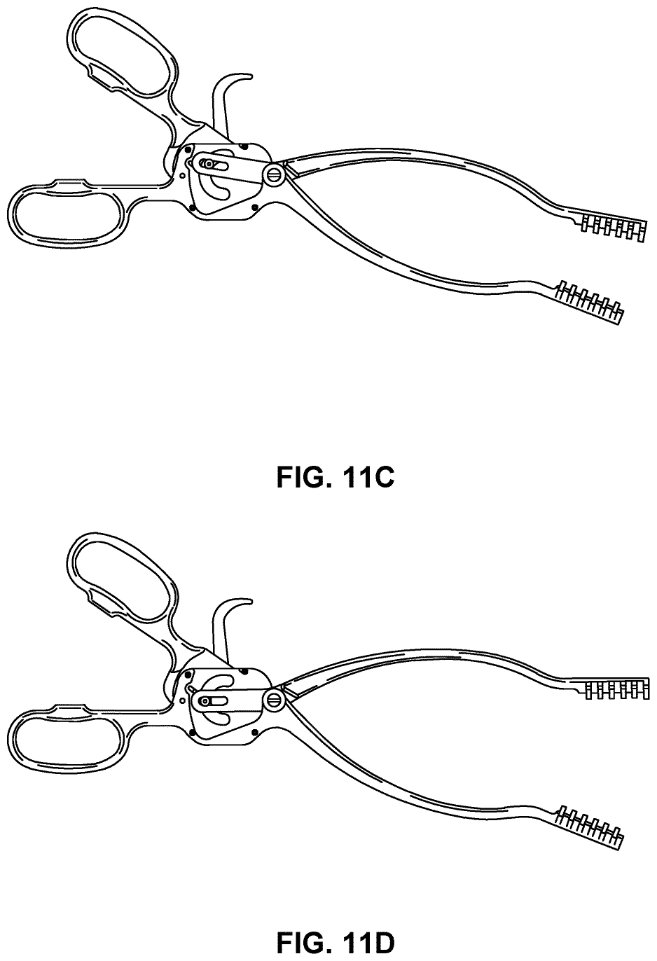

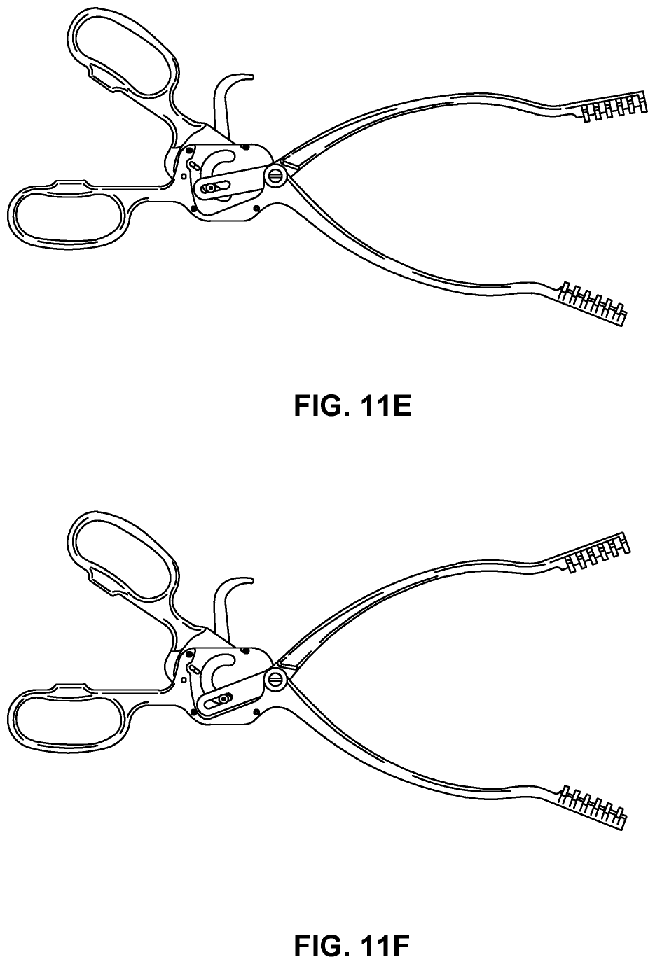

FIG. 11a is an illustration of a second retractor embodiment in a fully closed state and expanded handle configuration. In this alternative embodiment, many of the same components are present as described above within the retractor 200. Once again, the first arm 216 is a unitary element from proximal end 214 to distal end 218. The second handle arm 232 is pivotally attached to and rotatable relative to the proximal section 220 of the first arm 216 at pivot HP. This pivot HP is spaced proximally from the pivot RP for the distal section of the first arm 216 and the second retractor arm 236.

In this embodiment, the protrusion 244 that extends from the first away from the pivot RP and applies leverage to the second retractor arm 236 relative to the first arm 216 during spreading, also serves as the equivalent of the link element 142 in the previous embodiment. The end of the protrusion 244 is provided with an elongate slot 270 that cooperates with a pin 272 connected to the mechanism 226. The pin 272 is confined within and moves within a curved channel 252.

FIG. 11b is an illustration of the FIG. 11a embodiment in a first intermediate state and expanded handle configuration; providing a first spreading gap.

FIG. 11c is an illustration of the FIG. 11a embodiment in a second intermediate state and expanded handle configuration; providing a second increased spreading gap.

FIG. 11d is an illustration of the FIG. 11a embodiment in a third intermediate state and expanded handle configuration; providing a third further increased spreading gap. FIG. 11e is an illustration of the FIG. 11a embodiment in a fourth intermediate state and expanded handle configuration; providing a fourth further increased spreading gap.

FIG. 11f is an illustration of the FIG. 11a embodiment in a fully open state and expanded handle configuration; providing a fifth and largest spreading gap.

Once again, as the spreading gaps increase, the pin 244 moves within the channel 252 from one end to the other.

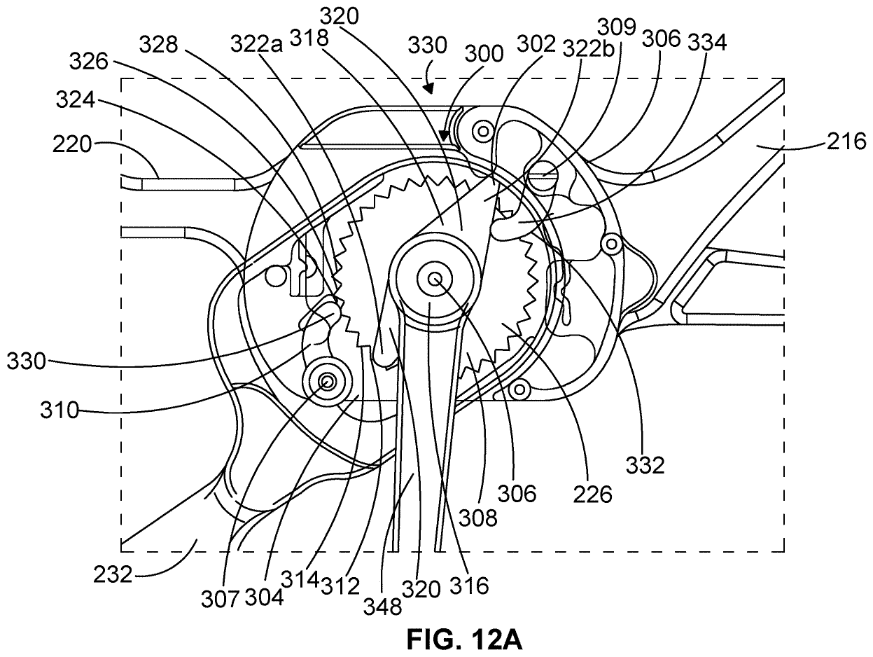

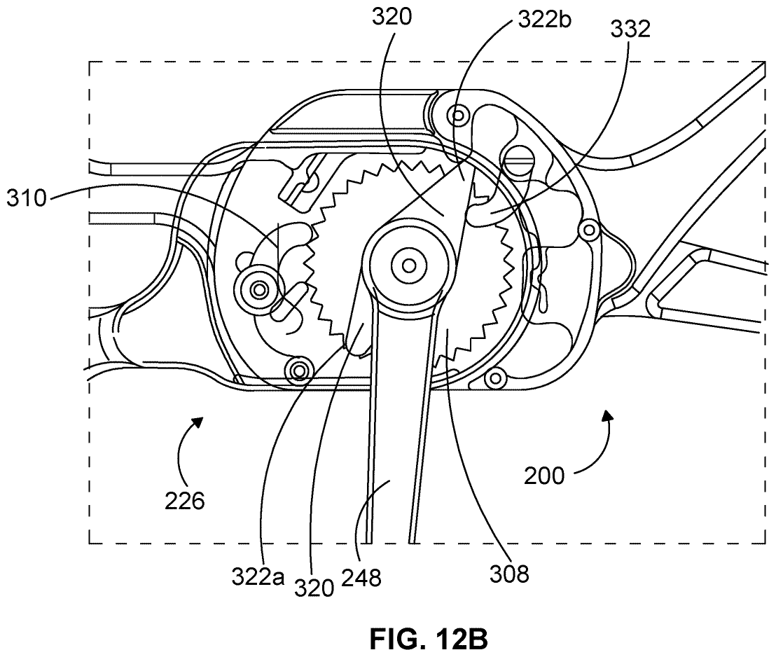

FIG. 12a is a detailed view of the mechanism 226 of the FIG. 11a to 11f embodiment from a first side in the expanded handle configuration, whilst FIG. 12b is a detailed view from the same side in the contracted handle configuration. Whilst this mechanism 226 is described in the context of the second embodiment, a similar mechanism with minor modification can be used in the first embodiment.

Referring to FIG. 12a, the mechanism 226 is provided within a mechanism housing 300 formed of a first mechanism housing part 302 mounted on the proximal section 220 of the first arm 216 and a second mechanism housing part 304 mounted on the second handle arm 232. The first mechanism housing part 302 and the second mechanism housing part 304 combine to enclose the mechanism 226.

In FIG. 12a, the second handle arm 232 is seen closest to the viewer and is mounted on a pivot 306 which extends into the mechanism 226. Within the second mechanism housing part 304, the pivot 306 also mounts a toothed cog wheel 308.

Inside the second housing part 304, a first pawl 310 is mounted on the second housing part 304, such that the first pawl 310 moves with the second handle arm 232 when that rotates. The first pawl 310 is pivotally mounted on the second mechanism housing part 304 at pivot 307 and is provided with a leaf spring to urge the first pawl 310 into engagement with the recesses 312 between the cogs 314 on the cog wheel 308. The first pawl 310 is curved towards the cog wheel 308.

The release lever 248 is fixed to a collar 316 which extends into the mechanism 226 and the collar 316 is rotatably mounted on the pivot 306. The collar 316 is also fixedly mounted on a plate 318 which provide a pair of opposing projections 320 that each end in a cam 322a, 322b. The operation of the release lever 248 is described in further detail below. The plate 318 is provided between the inside of the second mechanism housing part 304 and the toothed cog wheel 308.

Referring to FIG. 12a, when the second handle arm 232 is initially rotated relative toward the proximal end 220 of the first arm 216, the first pawl 310 is moved clockwise up and over the top of the first cog 324 on the cogwheel 308. The force applied by the leaf spring then causes the first pawl 310 to enter the next recess 326 between the first cog 324 and the next cog 328. Furthermore, the orientation of the first pawl 310 [curved towards the recess 326] and the shape of the recess 326 and the end 330 of the first pawl 310 cause the first pawl 310 to engage with the cogwheel 308 such that further rotation of the second handle arm 232 also causes rotation of the cogwheel 308. As such, rotation of the second handle arm 232 directly results in a matching rotation of the toothed cog wheel 308. This rotation continues until the contracted handle configuration is reached.

On the other side of the mechanism 226 to the first pawl 310, a second pawl 332 is also pivotally mounted but on the first mechanism housing part 302 at pivot 309 and is provided with a leaf spring to urge the second pawl 332 into engagement with the recesses 334 between the cogs 336 on the cog wheel 308. The second pawl 332 is also curved towards the cog wheel 308, but in the opposite direction to the first pawl 310. As the cogwheel 308 rotates, pushed by the first pawl 310, the relative shapes of the second pawl 332 and cogs 336 on the cog wheel 308 cause the second pawl 332 to ride up and over the cogs 336, with the leaf spring encouraging the second pawl 332 back into the recesses 334 when the cog 336 has rotated past. The second pawl 332 thus allows clockwise rotation of the cogwheel 308 in FIG. 12a.

When the second handle arm 232 and proximal end 220 of the first arm 216 reach the closed handle configuration, the second handle arm 232 can be transitioned back to the expanded handle state. As the second handle arm 232 rotates relative to the proximal section 220 of the first arm 216, the first pawl 310 direction of curve and the relative shape of the cogs 324, 328, recesses 326 and end of the first pawl 310 all allow free movement of the second handle arm 232 and the first pawl 310 anticlockwise relative to the cogwheel 308. The second pawl 332 resists the cogwheel 308 rotating in the anticlockwise direction in FIG. 12a. Thus, the second pawl 332 operations to maintain the position of the distal end 220 of the first arm 216 and the second retractor arm 232, and hence the value of the spreading gap for the retractor elements whilst the second handle arm 232 and proximal section 220 of the first arm 216 are transitioned from the closed handle configuration to the expanded handle configuration.

Once in the expanded handle configuration, a further transition cycle to the contracted handle configuration can be used to cause rotation of the cogwheel 308 in the clockwise direction in FIG. 12a.

As seen in FIG. 11a, on the other side of the toothed cogwheel 308 and extending from the side surface 340 of the cogwheel 308 is pin 272. The pin 272 extends through a channel 252 in the first mechanism housing part 304. The side surface 340 of the cogwheel 308 is visible at the bottom of the channel 252. As the cogwheel 308 is rotated, so the pin 272 rotates with it. The pin 272 is engaged with the side of elongate slot 270 in the proximal end 244 of the second retractor arm 236. Thus, the pin 272 pushes the proximal end 244 of the second retractor arm 236 in the same direction and by virtue of the pivot RP this causes the second retractor arm 236 to move in the opposite direction and so increase the spreading gap 238.

The movement of the pin 272 continues which the second handle arm 232 rotates and hence the cogwheel 308 rotates; anticlockwise movement of the second handle arm 232 in FIG. 11a. During a single cycle of the expanded handle configuration to contracted handle configuration change the pin 272 advances along only a part of the channel 252.

When the second handle arm 232cis rotated clockwise in FIG. 11a, to reach the expanded handle configuration, then the cogwheel 308 and the pin 272 mounted upon it stay locked in the same position and hence the force applied to the proximal end 244 of the second retractor arm 236 is maintained.

Further pumps of the handles cause further progression of the pin 272 and further increases in the spreading gap 238, as shown in the FIG. 11a to 11f sequence.

Referring to FIG. 12b, when it is desired to release the retractor 200, the surgeon moves the end of the release lever 248 proximally and this causes the release lever 248 to rotate as the release lever 248 is pivotally mounted. That rotation in turn causes rotation of the two opposing projections 320 mounted on the release lever 248 inside the casing. The ends of the projections 320 each act as a cam 322a, 322b, which rotate into abutment with the first pawl 310 and second pawl 332 of the mechanism 226. Further rotation of the cams 322a, 322b causes them to push the first pawl 310 and the second pawl 332 out of engagement with the recesses in the cogwheel 308. The cogwheel 308 is then free to rotate and the tension at the incision site causes the retractor elements to close. This allows for repositioning of the retractor 200 and/or the disengagement of the retractor 200 from the patient.

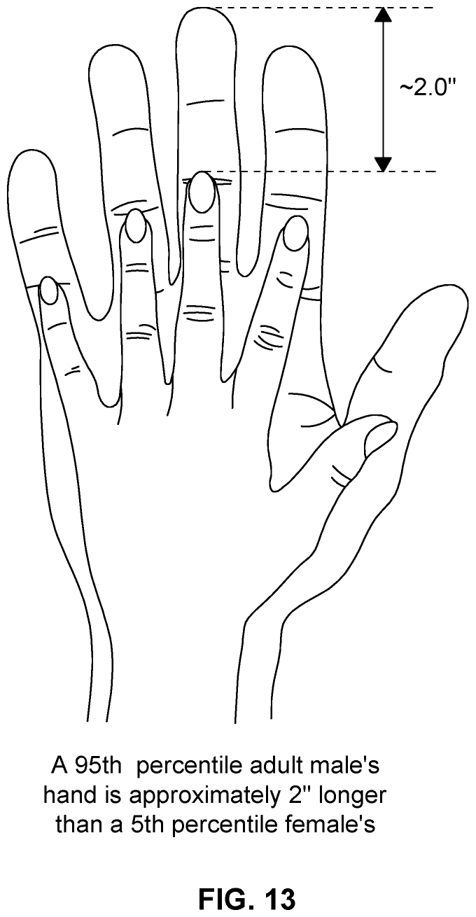

As illustrated in FIG. 13, there are material size differences between hands of surgeons. In this case the 95^{th} percentile hand dimensions for a male surgeon and the 5^{th}percentile hand dimensions for a female surgeon are shown. By using multiple pump action type activation over multiple cycles, rather than a single actuation, the retractor design achieves the desired level of retraction without a grip range of movement and gip force requirement that is within comfortable achievement by a larger number of surgeons.

Other approaches, such as shortening the proximal section handle gap that needs to be closed might bring that aspect within the range of more surgeons, but that style of approach would necessitate an increase retractor arm length at the other end to achieve the desired spread and that would also increase the force needed as the lever length is reduced.

Within the disclosure, the length of the retractor arms [the extent away from the pivot RP] is configured to give the desired maximum spreading gap value needed. This could be at least 120 mm, for instance at least 130 mm and potentially at least 135 mm. The maximum spreading gap may be less than 150 mm, for instance less than 140 mm.

In general, the angular separation between the two retractor arms varies between zero, such as zero to 5°, in the closed state up to 40° in the fully opened state that delivers the desired spreading gap.

The first ring grip and second ring grip may have at least a spacing of 2 mm between their closest points in the contracted handle configuration. The maximum distance may be 60 mm or less, for instance 56 mm +/−3%.

In providing these aims, the overall retractor can still be operated fully without the need for high force levels applied to the handles. Typically, the maximum grip force would be 15 N and the maximum force needed repetitively would be 10 N.

At the same time, these force limits can be obtained whilst also keeping the maximum handle spread used within acceptable values, for instance a grip span of 10 cm and avoiding any span above 13 cm.

As described above, the mechanism 226 is configured to give a linear response; a fixed rotation per pump of the handles is employed. It would be possible to swap to a fixed force applied to the handles resulting in a lower rotation with each progressive pump, as more resistance is encountered; a non-linear response.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. A surgical instrument, comprising
a first force input element and a second force input element, the first force input element and the second force input element being pivotally mounted relative to one another by a pivot, the first force input element being provided on a first side of the instrument and being provided in opposition to the second force input element provided on the second side of the instrument;
a first force output element and a second force output element, the first force output element and the second force output element being pivotally mounted relative to one another by a pivot, the second force output element being provided on the first side of the instrument and being provided in opposition to the first force output element provided on the second side of the instrument.

2. The surgical instrument of claim 1, wherein the first force input element and the first force output element define a unitary element and the first force input element and the first force output element have a fixed position for the first force input element relative to the first force output element.

3. The surgical instrument of claim 1, wherein the second force input element and the second force output element define separate elements and the second force input element and the second force output element have a non-fixed position for the second force input element relative to the second force output element.

4. The surgical instrument of claim 1, wherein the pivot for the first force input element and the second force input element is a different pivot to the pivot for the first force output element and the second force output elements.

5. The surgical instrument of claim 1, wherein the pivot for the first force input element and the second force input element is a first pivot and the pivot for the first force output element and the second force output elements is a second pivot, and wherein the first pivot is spaced apart from the second pivot.

6. The surgical instrument of claim 1, wherein the instrument has a proximal end at the force input element end and the instrument has a distal end at the force output element end, wherein the first pivot for the first force input element and the second force input element is provided closer to the proximal end of the instrument than the second pivot for the first force output element and the second force output element.

7. The surgical instrument of claim 1, according to any preceding claim wherein the first pivot is spaced from the second pivot by a distance of greater than 2 cm and less than 10 cm.

8. The surgical instrument of claim 1, wherein the instrument further comprises a mechanism between at least one of the force input elements and at least one of the force output elements.

9. The surgical instrument of claim 1, wherein the instrument further comprises a mechanism between only one of the force input elements and only one of the force output elements.

10. The surgical instrument of claim 4, wherein a mechanism defines a link between the first pivot and the second pivot and the link conveys force inputted about the first pivot to force outputted about the second pivot.

11. The surgical instrument of claim 8, according wherein the mechanism comprises a first part of the mechanism and a second part of the mechanism, the second part of the mechanism is provided by the second force input element by a second mechanism part housing and a second drive element which conveys a drive force to the first part of the mechanism.

12. The surgical instrument of claim 11, wherein the first part of the mechanism comprises the first force input element and a first drive element which conveys a drive force from a second part of the mechanism to an interface with the second arm.

13. The surgical instrument of claim 12, wherein the first drive element conveys the drive force through a pin extending from the first drive element.

14. The surgical instrument of claim 13, wherein the pin extends through a channel in the first mechanism part housing.

15. (canceled)

16. (canceled)

17. The surgical instrument of claim 8, wherein the mechanism includes a first locking element, the first locking element maintaining the instrument in a given closed state and/or intermediate state and/or open state.

18. The surgical instrument of claim 17, wherein the first locking element engages with a first drive element to prevent rotational movement of the first drive element.

19. The surgical instrument of claim 1, further comprising a release mechanism including a rotatable arm and a release plate including one or more protrusions, the release mechanism being adapted to move the first locking element out of cooperation with the first drive element and/or adapted to move the second drive element out of cooperation with the first drive element.

20. (canceled)

21. (canceled)
